(19) United States
(12) Patent Application Publication
(10) Pub. No.: US 2012/0031047 A1
(43) Pub. Date: Feb. 9, 2012

(54) METHOD FOR STORING TRANSDERMALLY/TRANSMUCOSALLY ABSORBABLE PREPARATION AND PACKAGE OF TRANSDERMALLY/TRANSMUCOSALLY ABSORBABLE PREPARATION

(75) Inventors: Tomohiro SHINODA, Tsukuba-shi (JP); Takashi Yasukouchi, Tsukuba-shi (JP); Yasunori Takada, Tsukuba-shi (JP)

(73) Assignee: HISAMITSU PHARMACEUTICAL CO., INC., Tosu-shi (JP)

(21) Appl. No.: 13/196,389

(22) Filed: Aug. 2, 2011

(30) Foreign Application Priority Data
Aug. 3, 2010 (JP) 2010-174159

(51) Int. Cl. B65B 31/00; C07C 271/44; C07C 211/27

(52) U.S. Cl. 53/403; 564/316; 560/163

(57) ABSTRACT

A method for storing a transdermally/transmucosally absorbable preparation, comprising keeping a transdermally/transmucosally absorbable preparation enclosed in a container in a low oxygen atmosphere, the transdermally/transmucosally absorbable preparation comprising a drug whose molecule has an amino group substituted with a lower alkyl group.

### BACKGROUND OF THE INVENTION

1. Field of the Invention

The present invention relates to a method for storing a transdermally/transmucosally absorbable preparation, and a package of a transdermally/transmucosally absorbable preparation, wherein the transdermally/transmucosally absorbable preparation is placed in a container.

2. Related Background Art

Conventionally, transdermally/transmucosally absorbable preparations for administering active ingredients transdermally/transmucosally have been developed from the viewpoints of reducing adverse effects associated with oral administration, and of improving the quality of life of patients.

For example, International Application Japanese-Phase Publication No. 2002-523446 (Document 1) describes a transdermal patch for transdermally administrating salts, prodrugs, and metabolites of tolterodine. Moreover, International Application Japanese-Phase Publication No. 2002-544222 (Document 2) describes a transdermal therapeutic system (TTS) comprising a (meth)acrylate copolymer containing ammonium groups, at least one plasticizer, and up to 25% by mass tolterodine.

However, drugs whose molecules each have an amino group substituted with a lower alkyl group, such as tolterodine, have a common problem that the drugs are denatured, and the efficacies thereof are reduced, during a long-term storage. The stabilities overtime are still insufficient in the cases of the transdermal patch comprising tolterodine described in Document 1 and the transdermal therapeutic system (TTS) described in Document 2.

### SUMMARY OF THE INVENTION

The present inventors have conducted an earnest study to solve the above-described problem of the conventional technique. As a result, the present inventors have found that the reduction in the efficacy of drugs whose molecules each have an amino group substituted with a lower alkyl group, such as tolterodine, is caused by an effect of oxygen. Moreover, the present inventors also have found a problem that when an antioxidant generally used to suppress the denaturation of a drug due to oxygen is added to a pharmaceutical preparation, a dealkylated product of a drug whose molecule has an amino group substituted with a lower alkyl group is formed in the pharmaceutical preparation over time, and the drug is inactivated.

The present invention has been made in view of the above-described problem, and an object of the present invention is to provide a method for storing a transdermally/transmucosally absorbable preparation comprising a drug whose molecule has an amino group substituted with a lower alkyl group and also to provide a package of the transdermally/transmucosally absorbable preparation. This storage method and this package make it possible to keep the drug in the transdermally/transmucosally absorbable preparation stable for a long period of time, while the formation of a dealkylated product, which may cause the inactivation of the drug, can be sufficiently suppressed.

The present inventors have conducted earnest study to achieve the above object. As a result, the present inventors have found that the above-described object can be achieved by keeping a transdermally/transmucosally absorbable preparation enclosed in a container in a low oxygen atmosphere, the transdermally/transmucosally absorbable preparation comprising a drug whose molecule has an amino group substituted with a lower alkyl group. This finding has led to the completion of the present invention.

Specifically, a method for storing a transdermally/transmucosally absorbable preparation of the present invention comprises keeping a transdermally/transmucosally absorbable preparation enclosed in a container in a low oxygen atmosphere, the transdermally/transmucosally absorbable preparation comprising a drug whose molecule has an amino group substituted with a lower alkyl group.

Meanwhile, a package of a transdermally/transmucosally absorbable preparation of the present invention comprises: a transdermally/transmucosally absorbable preparation comprising a drug whose molecule has an amino group substituted with a lower alkyl group; and a container whose inside atmosphere is a low oxygen atmosphere and in which the transdermally/transmucosally absorbable preparation is enclosed.

In the method for storing a transdermally/transmucosally absorbable preparation of the present invention and the package of a transdermally/transmucosally absorbable preparation of the present invention, tolterodine or rivastigmine is preferably used as the drug whose molecule has an amino group substituted with a lower alkyl group.

In addition, in the method for storing a transdermally/transmucosally absorbable preparation of the present invention and the package of a transdermally/transmucosally absorbable preparation of the present invention, an oxygen concentration in the container is preferably 3.0% by volume or less.

Moreover, in the method for storing a transdermally/transmucosally absorbable preparation of the present invention and the package of a transdermally/transmucosally absorbable preparation of the present invention, the transdermally/transmucosally absorbable preparation is preferably one selected from the group consisting of an ointment, a cream, a gel, a lotion, a spray, and a patch.

The present invention makes it possible to provide a method for storing a transdermally/transmucosally absorbable preparation comprising a drug whose molecule has an amino group substituted with a lower alkyl group and a package of the transdermally/transmucosally absorbable preparation. This storage method and this package make it possible to keep the drug in the transdermally/transmucosally absorbable preparation stable for a long period of time, while the formation of a dealkylated product, which may cause the inactivation of the drug, can be sufficiently suppressed.

### DETAILED DESCRIPTION OF THE PREFERRED EMBODIMENTS

Hereinafter, the present invention will be described on the basis of preferred embodiments thereof.

First, a method for storing a transdermally/transmucosally absorbable preparation of the present invention will be described. The method for storing a transdermally/transmucosally absorbable preparation of the present invention comprises keeping a transdermally/transmucosally absorbable preparation enclosed in a container in a low oxygen atmosphere, the transdermally/transmucosally absorbable preparation comprising a drug whose molecule has an amino group substituted with a lower alkyl group. Hereinafter, the transdermally/transmucosally absorbable preparation to which the storage method of the present invention is applied and the container used in the present invention are first described, and then the storage method of the present invention is described.

(Transdermally/Transmucosally Absorbable Preparation)

The transdermally/transmucosally absorbable preparation to which the storage method of the present invention is applied comprises a drug whose molecule has an amino group substituted with a lower alkyl group.

Examples of the lower alkyl group include linear, branched, or cyclic alkyl groups having 1 to 6 carbon atoms, and examples of the alkyl groups include a methyl group, an ethyl group, a propyl group, an isopropyl group, a cyclopropyl group, a n-butyl group, an isobutyl group, a tert-butyl group, a cyclobutyl group, a n-pentyl group, a 2-methyl-butyl group, a 3-methyl-butyl group, a neopentyl group, a cyclopentyl group, a n-hexyl group, a 2-methyl-pentyl group, a 3-methyl-pentyl group, and a cyclohexyl group. Of these lower alkyl groups, linear, branched, or cyclic alkyl groups having 1 to 3 carbon atoms such as a methyl group, an ethyl group, a propyl group, an isopropyl group, and a cyclopropyl group are preferable. The amino group substituted with a lower alkyl group may be an amino group whose one hydrogen atom is substituted with the lower alkyl group, or an amino group whose two hydrogen atoms are each substituted with the lower alkyl group.

The drug whose molecule has an amino group substituted with a lower alkyl group is preferably basic. As the drug, tolterodine(4-methyl-2-[(R)-3-(diisopropylamino)-1-phenylpropyl]phenol) and rivastigmine (methylethylcarbamic acid 3-[(S)-1-(dimethylamino)ethyl]phenyl ester) are particularly preferable.

The dosage form of the transdermally/transmucosally absorbable preparation is not particularly limited, as long as the preparation in the dosage form enables the drug to be absorbed transdermally or transmucosally through the skin or the mucous membrane. Examples of the dosage form include an ointment, a cream, a gel, a lotion, a spray, and a patch such as a hydrogel patch or a plaster. Hereinafter, the patch will be described as a preferred example of the transdermally/transmucosally absorbable preparation to which the storage method of the present invention is applied.

One example of the patch is a patch comprising a support and an adhesive agent layer formed on one surface of the support.

The support is not particularly limited, as long as the support is capable of supporting the adhesive agent layer. The support preferably has a moderate flexibility, from the viewpoint of enhancing the adhesiveness of the patch to the skin. Examples of such a support include plastic films, woven fabrics, and nonwoven fabrics. Examples of materials of the plastic films, the woven fabrics, and the nonwoven fabrics include resins of polymers such as polyesters, polypropylene, polyethylene, polyvinyl acetate, and polyvinyl chloride, and resins of polymers obtained by co-polymerization of monomers constituting any of these polymers (ethylene-vinyl acetate copolymer and the like). The thickness of the support is not particularly limited, and is preferably about 2 to 3000 μm, in general.

The adhesive agent layer comprises the drug and an adhesive agent. The thickness of the adhesive agent layer is not particularly limited, and is preferably about 10 to 300 μm in general.

In the patch to which the storage method of the present invention is applied, the drug contained in the adhesive agent layer is the drug whose molecule has an amino group substituted with a lower alkyl group. The content of the drug in the adhesive agent layer is preferably 0.05 to 50% by mass.

The adhesive agent is preferably one which is safe to the skin, and has adhesiveness enough to fix the patch to the skin surface at normal temperature. Any generally known adhesive agent used for a patch can be used as the adhesive agent. An example of the adhesive agent is an adhesive agent comprising a base, a tackifier, and a softener. Examples of the base include natural rubber-based materials, synthetic rubber-based materials, acrylic-based resin materials, silicone-based resin materials, and the like. Of these bases, synthetic rubber-based materials and acrylic-based resin materials are preferable from the viewpoints of excellent adhesiveness and excellent ability to release the drug. Examples of the synthetic rubber-based materials include homopolymers such as polyisobutylene and polyisoprene, and copolymers containing any of these polymers. Of these synthetic rubber-based materials, polyisobutylene and styrene-isoprene-styrene block copolymers are preferable. Examples of the acrylic-based resin materials include homopolymers of (meth)acrylic acids/esters such as acrylic acid, 2-ethylhexyl acrylate, methyl acrylate, butyl acrylate, hydroxyethyl acrylate, and 2-ethylhexyl methacrylate; and copolymers containing at least one of these (meth)acrylic acids/esters (2-ethylhexyl acrylate.vinyl acetate copolymers, 2-ethylhexyl acrylate.vinyl acetate.acrylic acid copolymers, 2-ethylhexyl acrylate.vinyl acetate.hydroxyethyl acrylate copolymers, 2-ethylhexyl acrylate.vinyl acetate.hydroxyethyl acrylate.acrylic acid copolymers, 2-ethylhexyl acrylate.2-ethylhexyl methacrylate.dodecyl methacrylate copolymers, and the like). Of these acrylic-based resin materials, 2-ethylhexyl acrylate.vinyl acetate copolymers and 2-ethylhexyl acrylate.vinyl acetate.acrylic acid copolymers are preferable. In addition, one of or a combination of two or more of these bases may be used as the base.

Examples of the tackifier include alicyclic saturated hydrocarbon resins, rosin derivatives (rosin, rosin glycerin ester, hydrogenated rosin, hydrogenated rosin glycerin ester, rosin pentaerythritol ester, and the like), terpene resins, petroleum resin, and maleic acid resins. Of these tackifiers, alicyclic saturated hydrocarbon resins and hydrogenated rosin ester are preferable. In addition, one or a combination of two or more of these tackifiers may be used as the tackifier.

Examples of the softener include petroleum-based oils (paraffin-based process oils, naphthene-based process oils, aromatic-based process oils, and the like), squalane, squalene, vegetable-based oils (almond oil, olive oil, camellia oil, castor oil, tall oil, peanut oil, and the like), olefinic acids, silicone oils, diprotic acid esters (dibutyl phthalate, dioctyl phthalate, and the like), liquid rubbers (liquid polybutene, liquid polyisoprene, and the like), liquid fatty acid esters (isopropyl myristate, hexyl laurate, diethyl sebacate, isopropyl sebacate, and the like), diethylene glycol, polyethyleneglycol, glycol salicylate, propylene glycol, dipropylene glycol, triacetin, triethyl citrate, and crotamiton. Of these softeners, liquid paraffin, isopropyl myristate, and diethyl sebacate are preferable, from the viewpoint that a moderate adhesiveness to the skin can be provided. In addition, one of or a combination of two or more of these softeners can be used as the softener.

In the patch to which the storage method of the present invention is applied, the adhesive agent layer may further comprise additives such as an antioxidant, a filler, a cross-linking agent, a preservative, and an ultraviolet absorber, if necessary. As the antioxidant, tocopherols, ester derivatives thereof, ascorbic acid, ascorbyl stearate, nordihydroguaiaretic acid, dibutylhydroxytoluene, and butylhydroxyanisole are preferable. As the filler, calcium carbonate, magnesium carbonate, silicates (aluminum silicates, magnesium silicates, and the like), silicic acid, barium sulfate, calcium sulfate, calcium zincate, zinc oxide, titanium oxide are preferable. As the cross-linking agent, thermosetting resins such as amino resins, phenol resins, epoxy resins, alkyd resins, and unsaturated polyesters; isocyanate compounds; block isocyanate compounds; organic cross-linking agents; and inorganic cross-linking agents such as metals and metal compound are preferable. As the preservative, ethyl paraben, propyl paraben, and butyl paraben are preferable. As the ultraviolet absorber, p-aminobenzoic acid derivatives, anthranilic acid derivatives, salicylic acid derivatives, coumarin derivatives, amino acid-based compounds, imidazoline derivatives, pyrimidine derivatives, and dioxane derivatives are preferable. The content of such additives in the adhesive agent layer is preferably 10% by mass or less, more preferably 5% by mass or less, and particularly preferably 2% by mass or less in the adhesive agent layer.

A method for producing the patch to which the storage method of the present invention is applied is not particularly limited, and a known method for producing a patch can be employed appropriately. For example, the patch can be produced in such a manner that an adhesive agent layer is formed by spreading an adhesive agent layer composition obtained by mixing the drug and the adhesive agent on the support.

Hereinabove, the patch is described as a preferred example of the transdermally/transmucosally absorbable preparation to which the storage method of the present invention is applied. However, the transdermally/transmucosally absorbable preparation to which the storage method of the present invention is applied is not limited to the patch. The transdermally/transmucosally absorbable preparation only needs to comprise a drug whose molecule has an amino group substituted with a lower alkyl group, and may be in the form of, for example, an ointment, a cream, a gel, a lotion, a spray, or the like.

(Container)

The container used in the present invention is preferably one having a low oxygen permeability from the viewpoint of maintaining a low oxygen atmosphere. The oxygen permeability is preferably 100 cm³/(m²·24 hours·atm) or less, and more preferably 15 cm³/(m²·24 hours·atm) or less.

Examples of materials of such a container include metal foils such as aluminum foil; films having a low oxygen permeability such as ethylene-vinyl alcohol copolymer films, plastic films on which a metal (aluminum or the like) is deposited, and plastic films on which a ceramic (silicon oxide or the like) is deposited; metals such as stainless steel; and glass. Of these materials, aluminum foil is preferably used because the oxygen permeability of the container tends to be further reduced, and light and moisture tend to be blocked. In addition, one of or a combination of two or more of these materials may be used, and these materials may be used in combination with any other materials. For example, the aluminum foil or the film having a low oxygen permeability may be used in the form of a layered film formed with a polyacrylonitrile film, a polyethylene film, cellophane, and the like. Alternatively, the aluminum foil or the film having a low oxygen permeability may be used in the form of a layered film further comprising a layer containing an oxygen absorber to be described later.

The size of the container may be selected appropriately depending on the transdermally/transmucosally absorbable preparation. Moreover, the container may have any shape, as long as the transdermally/transmucosally absorbable preparation can be enclosed in the container. The container may be a pouch-shaped container or a molded container.

(Method for Storing Transdermally/Transmucosally Absorbable Preparation)

The storage method of the present invention is a method comprising keeping the transdermally/transmucosally absorbable preparation enclosed in the container in a low oxygen atmosphere.

The present invention needs to be such that the transdermally/transmucosally absorbable preparation is enclosed in the container, while the inside atmosphere of the container is kept in a low oxygen atmosphere. The low oxygen atmosphere refers to an atmosphere having an oxygen concentration of 3.0% by volume or less. If the oxygen concentration exceeds the upper limit, the drug whose molecule has an amino group substituted with a lower alkyl group in the transdermally/transmucosally absorbable preparation is dealkylated over time into an inactive dealkylated product, so that the efficacy of the drug is reduced. In addition, the oxygen concentration is preferably 1.0% by volume or less, and more preferably 0.5% by volume or less from the same viewpoints as above, because a higher effect tends to be obtained.

In the enclosure method of the present invention, it is possible to first establish the low oxygen atmosphere in the container, and then enclose the transdermally/transmucosally absorbable preparation, or it is also possible to first enclose the transdermally/transmucosally absorbable preparation, and then establish the low oxygen atmosphere in the container. Examples of the method include a method in which the pressure inside the container is first reduced, and then the transdermally/transmucosally absorbable preparation is enclosed; a method in which the air in the container is replaced with an inert gas such as nitrogen gas or argon gas, and then the transdermally/transmucosally absorbable preparation is enclosed; a method in which both the transdermally/transmucosally absorbable preparation and an oxygen absorber are enclosed in the container; and the like. One of or a combination of two or more of these methods may be used. For example, a method may be employed in which both the transdermally/transmucosally absorbable preparation and the oxygen absorber are enclosed in a container subjected to nitrogen gas replacement.

When the oxygen absorber is used, a generally used oxygen absorber or also a commercially available oxygen absorber can be used appropriately as the oxygen absorber. The oxygen absorber may be of a self reaction type or a water dependent type, and may be an inorganic oxygen absorber containing iron powder, zinc powder, hydrosulfite, or the like as a base agent; or an organic oxygen absorber such as ascorbic acid-based oxygen absorber, a polyalcohols-based oxygen absorber, an activated carbon-based oxygen absorber, or the like. Of these oxygen absorbers, those which absorb oxygen in shorter days and hence which are quick-acting are preferable. As the oxygen absorber, for example, any of the following oxygen absorbers can be used directly or in a packaged state: PharmaKeep (manufactured by Mitsubishi Gas Chemical Company, Inc.), AGELESS (manufactured by Mitsubishi Gas Chemical Company, Inc.), StabilOx (Manufactured by Multisorb Technologies), WELL PACK (manufactured by TAISEI Co., Ltd.), Ever Fresh (manufactured by Torishige Sangyo Co., Ltd.), Oxy-Eater (manufactured by Ueno Fine Chemicals Industry, Ltd.), KEEPIT (manufactured by Dorency Co., Ltd.), KEPLON (manufactured by Keplon Co., Ltd.), SANSO-CUT (manufactured by Iris fineproducts Co., Ltd.), SANSORESU (manufactured by Hakuyo, inc.), Sequl (manufactured by Nisso Jushi Co., Ltd.), TAMOTSU (manufactured by OhE Chemicals Inc.), VITALON (manufactured by Tokiwa Sangyo), Modulan (manufactured by Nippon Kayaku Food Techno Co., Ltd.), Wonder Keep (manufactured by Powder Tech), and Keep Fresh Type C (manufactured by Toppan Printing Co., Ltd.). In addition, the mass of the oxygen absorber used can be adjusted appropriately depending on the mass of the patch, the material and the capacity of the container, and the like. The mass is preferably such that the amount of oxygen absorbed by the oxygen absorber is 2.0 μL or more.

The degree of the enclosure is preferably such that the container is sealed so tightly that no oxygen can permeate into the container. The enclosure method can be selected appropriately depending on the container. For example, a heat seal method may be employed when a pouch-shaped container made of a plastic onto which aluminum is deposited is used.

(Package of Transdermally/Transmucosally Absorbable Preparation)

Next, a package of a transdermally/transmucosally absorbable preparation of the present invention will be described. The package of a transdermally/transmucosally absorbable preparation of the present invention comprises a transdermally/transmucosally absorbable preparation comprising a drug whose molecule has an amino group substituted with a lower alkyl group; and a container whose inside atmosphere is a low oxygen atmosphere and in which the transdermally/transmucosally absorbable preparation is enclosed.

The transdermally/transmucosally absorbable preparation according to the present invention comprises a drug whose molecule has an amino group substituted with a lower alkyl group. As the drug whose molecule has an amino group substituted with a lower alkyl group, the same drugs as described for the method for storing a transdermally/transmucosally absorbable preparation of the present invention can be used. Of those drugs, tolterodine(4-methyl-2-[(R)-3-(diisopropylamino)-1-phenylpropyl]phenol) and rivastigmine (methylethylcarbamic acid 3-[(S)-1-(dimethylamino)ethyl]phenyl ester) are particularly preferably used. In addition, the dosage form of the transdermally/transmucosally absorbable preparation according to the present invention is not particularly limited, and examples thereof include the same dosage forms described for the method for storing a transdermally/transmucosally absorbable preparation of the present invention.

The container according to the present invention is not particularly limited, as long as the inside atmosphere of the container can be kept in a low oxygen atmosphere, and the transdermally/transmucosally absorbable preparation can be enclosed in the container. As the container, the same containers as described for the method for storing a transdermally/transmucosally absorbable preparation of the present invention can be used. In addition, the low oxygen atmosphere is the same as described above.

A method for producing a package of a transdermally/transmucosally absorbable preparation of the present invention is not particularly limited. For example, the package of a transdermally/transmucosally absorbable preparation of the present invention can be obtained as follows. Specifically, the transdermally/transmucosally absorbable preparation is enclosed in the container whose inside atmosphere is a low oxygen atmosphere by employing the same method as the enclosure method described for the above-described method for storing a transdermally/transmucosally absorbable preparation.

### EXAMPLES

Hereinafter, the present invention will be described more specifically on the basis of Examples and Comparative Examples, but the present invention is not limited to the following Examples.

### Example 1

First, an adhesive agent layer composition was obtained by adding tolterodine tartrate (2.5 g) and sodium hydroxide (0.43 g: two molar equivalents relative to tolterodine tartrate)) to an OH group-containing acrylic adhesive agent (56.35 g: “DURO-TAK 87-4287” manufactured by Henkel AG & Co. KGaA).

Subsequently, an adhesive agent layer was formed by applying the obtained adhesive agent layer composition in a thickness of 100 μm onto a PET film (“Scotchpak 9732” manufactured by 3M Company). Thus, a patch was obtained in which the amount of tolterodine tartrate was 1 mg per cm² (10% by mass in the adhesive agent layer).

The obtained patch was placed together with an oxygen absorber.desiccant (“PharmaKeep KD-20” manufactured by Mitsubishi Gas Chemical Company, Inc.) in a pouch-shaped container (85 mm×80 mm) made of a multilayer film (a polyacrylonitrile film, aluminum foil, polyethylene, and cellophane), and then enclosed under conditions of a temperature of 23° C., an initial relative humidity of 38%, and an initial oxygen concentration of 20% by volume. The patch enclosed in the container was stored at a temperature of 60° C. for two weeks. Note that the oxygen concentration in the container four days after the enclosure was 0.1% by volume or less.

### Example 2

A patch obtained in the same manner as in Example 1 was stored in the same manner as in Example 1, except that an oxygen absorber (“AGELESS ZJ-PT” manufactured by Mitsubishi Gas Chemical Company, Inc.) was used instead of the oxygen absorber.desiccant. Note that the oxygen concentration in the container four days after the enclosure was 0.1% by volume or less.

### Example 3

First, an adhesive agent layer composition was obtained by adding rivastigmine (3.5 g) to a COOH group-containing acrylic adhesive agent (23.5 g: a 2-ethylhexyl acrylate.vinyl acetate.acrylic acid copolymer manufactured by Henkel AG & Co. KGaA).

Subsequently, an adhesive agent layer was formed by applying the obtained adhesive agent layer composition in a thickness of 60 μm onto a PET film (“Scotchpak 9732” manufactured by 3M Company). Thus, a patch was obtained in which the amount of rivastigmine was 1.8 mg per cm² (30% by mass in the adhesive agent layer).

The obtained patch was placed together with an oxygen absorber.desiccant (“PharmaKeep KD-20” manufactured by Mitsubishi Gas Chemical Company, Inc.) into a pouch-shaped container (85 mm×80 mm) made of a multilayer film (a polyacrylonitrile film, aluminum foil, polyethylene, and cellophane), and then enclosed under conditions of a temperature of 23° C., an initial relative humidity of 38%, and an initial oxygen concentration of 20% by volume. Then, the patch enclosed in the container was stored at a temperature of 60° C. for two weeks. Note that the oxygen concentration in the container four days after the enclosure was 0.1% by volume or less.

### Comparative Example 1

A patch obtained in the same manner as in Example 1 was enclosed in the same manner as in Example 1, except that no oxygen absorber.desiccant was used, and stored for two weeks under conditions of a relative humidity of 38%, an oxygen concentration of 20% by volume, and a temperature of 60° C.

### Comparative Example 2

A patch was obtained in the same manner as in Example 1, except that sodium sulfite (0.075 g) was added to the adhesive agent composition. The patch was enclosed in the same manner as in Example 1, except that no oxygen absorber.desiccant was used. The enclosed patch was stored for two weeks under conditions of a relative humidity of 38%, an oxygen concentration of 20% by volume, and a temperature of 60° C.

### Comparative Example 3

A patch was obtained in the same manner as in Comparative Example 2, except that sodium pyrosulfite (0.05 g) was added to the adhesive agent composition instead of sodium sulfite (0.075 g). The patch was stored in the same manner as in Comparative Example 2.

### Comparative Example 4

A patch was obtained in the same manner as in Comparative Example 2, except that ascorbic acid (0.075 g) was added to the adhesive agent composition instead of sodium sulfite (0.075 g). The patch was stored in the same manner as in Comparative Example 2.

### Comparative Example 5

A patch was obtained in the same manner as in Comparative Example 2, except that tocopherols (0.075 g) were added to the adhesive agent composition instead of sodium sulfite (0.075 g). The patch was stored in the same manner as in Comparative Example 2.

### Comparative Example 6

A patch was obtained in the same manner as in Comparative Example 2, except that dibutylhydroxytoluene (0.75 g) was added to the adhesive agent composition instead of sodium sulfite (0.075 g). The patch was stored in the same manner as in Comparative Example 2.

### Comparative Example 7

A patch obtained in the same manner as in Example 3 was enclosed in the same manner as in Example 3, except that no oxygen absorber.desiccant was used. The enclosed patch was stored for one month under conditions of a relative humidity of 38%, an oxygen concentration of 20% by volume, and a temperature of 60° C. When the patch stored for one month was subjected to high performance liquid chromatography, a peak different from that of rivastigmine was observed. This peak was analyzed with a LC/MS (Analyzer name: Q-T of Premier (JASCO International Co., Ltd.), measurement conditions: ESI mode, positive ions mode, a capillary voltage of 4.5 kV, a cone voltage of 20 V, and an ion source temperature of 150° C.). As a result, it was verified that a demethylated product, which is a dealkylated product of rivastigmine, was formed in the patch.

For each of the patches stored by the storage methods of Examples and Comparative Examples, the amount of a dealkylated product of tolterodine (the deisopropylated product: (+)-2-{(1R)-3-[(1-methylethyl)amino]-1-phenylpropyl}-4-methylphenol) formed, the amount of tolterodine remaining without dealkylation, and the amount of another dealkylated product (the demethylated product) of rivastigmine formed were measured by the following measurement methods, respectively.

<Measurement of Amounts of Dealkylated Products Formed>

(1) Measurement of Amount of Deisopropylated Product Formed

From each of the patches stored by the storage methods of Examples 1 to 2 and Comparative Examples 1 to 6, 6.25 cm² was taken as a sample. To the sample, 20 g of methanol was added, followed by stirring at room temperature for 5 hours. Thus, an extract of the drug was obtained. The obtained extract was measured for the deisopropylated product, which is one of the dealkylated products of tolterodine, by use of a high performance liquid chromatograph (manufactured by Shimadzu Corporation, column: an ODS column, solvent: phosphate buffer (0.2 mass/% by volume)/methanol=50/50 (volume ratio), detection wavelength: UV 210 nm). The ratio (% by mass) of the deisopropylated product formed to the mass of tolterodine added in the production of the patch (the initial amount of tolterodine added) was calculated by the following formula (1):

The amount of the deisopropylated product formed (% by mass)=the mass of the deisopropylated product (g)/the initial amount of tolterodine added (g)×100 (1),

and the obtained value was employed as the amount of the deisopropylated product formed.

(2) Measurement of Amount of Demethylated Product Formed

First, 10 cm² was taken as a sample from each of the patches stored by the storage methods of Example 3 and Comparative Example 7. To the sample, 30 ml of a purified water/methanol mixture solution (volume ratio: 1/1) was added, followed by extraction under reflux at 90° C. The extraction under reflux was repeated three times, and then a purified water/methanol mixture solution (volume ratio: 1/1) was further added. Thus, 200 ml an extract of the drug was obtained. Subsequently, the obtained extract was analyzed for the demethylated product by use of a high performance liquid chromatograph (manufactured by Shimadzu Corporation, column: an ODS column, solvent: a gradient mixture of a mixture solution of 10 mM ammonium acetate in purified water and acetonitrile (volume ratio: 95/5) and a mixture solution of 10 mM ammonium acetate in purified water and acetonitrile (volume ratio: 5/95), and detection wavelength: UV 215 nm). From the peak areas of rivastigmine, the demethylated product, and other unidentified substances detected in the measurement, the amount of the demethylated product formed (% by mass) was calculated by the following formula (2):

The amount of the demethylated product formed (% by mass)=the peak area of the demethylated product/(the sum of all the peak areas of rivastigmine, the demethylated product, and the other unidentified substances)×100 (2),

and the obtained value was employed as the amount of the demethylated product formed.

<Measurement of Amount of Remaining Tolterodine>

Each of the patches stored by the storage methods of Examples 1 to 2 and Comparative Examples 1 to 6 was measured for the amount of tolterodine in the same manner as in the measurement of the depropylated product. The ratio (% by mass) of tolterodine in the stored patch to the mass of tolterodine added in the production of the patch (the initial amount of tolterodine added) was calculated by the following formula (3):

The amount of remaining tolterodine (% by mass)=the mass of tolterodine in the stored patch (g)/the initial amount of tolterodine added (g)×100 (3),

and then the obtained value was employed as the amount of remaining tolterodine.

The following Table 1 shows the obtained results for the patches stored by the storage methods of Examples 1 to 3 and Comparative Examples 1 to 7. In Table 1, “Amount of dealkylated product formed” and “Amount of remaining drug” of each of Examples 1 to 2 and Comparative Examples 1 to 6 represent the ratio (% by mass) of the deisopropylated product to the initial amount of tolterodine added and the ratio (% by mass) of the amount of remaining tolterodine to the initial amount of tolterodine added, respectively. In addition, in Table 1, “Amount of dealkylated product formed” of each of Example 3 and Comparative Example 7 represents the ratio of the peak area of the demethylated product to the sum of all the peak areas detected by high performance liquid chromatography.

TABLE 1

| | | | | Amount of | Amount of |
|---|---|---|---|---|---|
| | | | Antioxidant | dealkylated | remaining |
| | Oxygen | | [Added amount | product formed | drug |
| | absorber | Desiccant | (mg/cm²)] | (% by mass) | (% by mass) |
| Example 1 | Present | Present | — | 0.22 | 100.02 |
| Example 2 | Present | — | — | 0.26 | 98.82 |
| Comparative | — | — | — | 0.74 | 88.6 |
| Example 1 | | | | | |
| Comparative | — | — | Sodium sulfite | 3.97 | 93.56 |
| Example 2 | | | [0.03] | | |
| Comparative | — | — | Sodium pyrosulfite | 3.64 | 95.35 |
| Example 3 | | | [0.02] | | |
| Comparative | — | — | Ascorbic acid | 1.68 | 94.31 |
| Example 4 | | | [0.03] | | |
| Comparative | — | — | Tocopherols | 1.27 | 94.51 |
| Example 5 | | | [0.03] | | |
| Comparative | — | — | Dibutylhydroxytoluene | 1.34 | 94.35 |
| Example 6 | | | [0.3] | | |
| Example 3 | Present | Present | — | 0.22 | — |
| Comparative | — | — | — | 0.80 | — |
| Example 7 | | | | | |

As is apparent from the results shown in Table 1, it was found, in each of the patches (Examples 1 to 2) stored by the method for storing a transdermally/transmucosally absorbable preparation of the present invention, that 98% by mass or more of tolterodine added in the production of the patch remained intact, and that the formation of the deisopropylated product was sufficiently suppressed. Note that, from the results of Examples 1 to 2, it was found that the effect of suppressing the formation of the deisopropylated product of the present invention was achieved by the low oxygen atmosphere rather than the dehumidification.

Meanwhile, although the amount of remaining tolterodine was slightly larger in each of the patches of Comparative Example 2 to 6 which contained the antioxidants and which were stored in a non-low oxygen atmosphere than in the case where no antioxidant was added (Comparative Example 1), the amount of the deisopropylated product formed was increased in each of Comparative Example 2 to 6. This showed that it was impossible to keep tolterodine in the pharmaceutical preparations of Comparative Example 2 to 6 stable for a long period of time.

Moreover, it was found that the formation of the demethylated product was sufficiently suppressed in the patch (Example 3) containing rivastigmine as the drug and stored by the method for storing a transdermally/transmucosally absorbable preparation of the present invention.

As described above, the present invention makes it possible to provide a method for storing a transdermally/transmucosally absorbable preparation comprising a drug whose molecule has an amino group substituted with a lower alkyl group and a package of the transdermally/transmucosally absorbable preparation. This storage method and this package make it possible to keep the drug in the transdermally/transmucosally absorbable preparation stable for a long period of time, while the formation of a dealkylated product, which may cause the inactivation of the drug, can be sufficiently suppressed.

Accordingly, the method for storing a transdermally/transmucosally absorbable preparation of the present invention is extremely useful in the pharmaceutical and medical industries.

What is claimed is:

1. A method for storing a transdermally/transmucosally absorbable preparation, comprising keeping a transdermally/transmucosally absorbable preparation enclosed in a container in a low oxygen atmosphere, the transdermally/transmucosally absorbable preparation comprising a drug whose molecule has an amino group substituted with a lower alkyl group.

2. The method for storing a transdermally/transmucosally absorbable preparation according to claim 1, wherein the drug whose molecule has an amino group substituted with a lower alkyl group is tolterodine or rivastigmine.

3. The method for storing a transdermally/transmucosally absorbable preparation according to claim 1, wherein an oxygen concentration in the container is 3.0%, by volume or less.

4. The method for storing a transdermally/transmucosally absorbable preparation according to claim 1, wherein the transdermally/transmucosally absorbable preparation is one selected from the group consisting of an ointment, a cream, a gel, a lotion, a spray, and a patch.

5. A package of a transdermally/transmucosally absorbable preparation, comprising:
a transdermally/transmucosally absorbable preparation comprising a drug whose molecule has an amino group substituted with a lower alkyl group; and
a container whose inside atmosphere is a low oxygen atmosphere and in which the transdermally/transmucosally absorbable preparation is enclosed.

6. The package of a transdermally/transmucosally absorbable preparation according to claim 5, wherein the drug whose molecule has an amino group substituted with a lower alkyl group is tolterodine or rivastigmine.

7. The package of a transdermally/transmucosally absorbable preparation according to claim 5, wherein an oxygen concentration in the container is 3.0% by volume or less.

8. The package of a transdermally/transmucosally absorbable preparation according to claim 5, wherein the transdermally/transmucosally absorbable preparation is one selected from the group consisting of an ointment, a cream, a gel, a lotion, a spray, and a patch.
